(19) United States
(12) Patent Application Publication
(10) Pub. No.: US 2024/0342665 A1
(43) Pub. Date: Oct. 17, 2024

(54) CELLULOSE NANOFIBER (CNF) STABILIZED MEMBRANES AND METHODS OF MAKING THEREOF

(71) Applicant: University of Maine System Board of Trustees, Orono, ME (US)

(72) Inventors: Michael Darin Mason, Orrington, ME (US); Muhammad Radowan Hossen, Orono, ME (US)

(21) Appl. No.: 18/294,825

(22) PCT Filed: Aug. 5, 2022
(86) PCT No.: PCT/US22/39575

§ 371 Date: Feb. 2, 2024

Related U.S. Application Data
(60) Provisional application No. 63/229,872, filed on Aug. 5, 2021.

(51) Int. Cl. B01D 71/10; B01D 67/00; B01D 69/02; B01D 69/14; B01D 71/74

(52) U.S. Cl. CPC B01D 71/10; B01D 67/003; B01D 69/02; B01D 69/14111; B01D 71/74; B01D 2311/14; B01D 2311/2688; B01D 2323/18; B01D 2323/20; B01D 2323/216; B01D 2323/21817; B01D 2323/21826; B01D 2325/40

(57) ABSTRACT

The present invention includes membranes comprising one or more cellulosic materials and wetting agent(s), and methods of making such membranes.

### CROSS REFERENCE TO RELATED APPLICATIONS

This application claims the benefit of U.S. Provisional Application No. 63/229,872, filed Aug. 5, 2021, the contents of which is hereby incorporated by reference.

### BACKGROUND

Membranes made of cellulosic materials (e.g., nitrocellulose) have been utilized in lateral flow devices, e.g., for diagnostics and other point of care devices (Mansfield 2005 Drugs of abuse. Ch. 4. pp. 71-85). However, such devices are limited in their wicking ability and methods of improving the dispersion of certain additives and optimizing wicking capabilities in cellulose-based membranes remains a challenge.

### SUMMARY OF THE INVENTION

Among other things, in some embodiments, the present invention provides compositions and membranes comprising one or more cellulosic materials (e.g., wood pulp and/or cellulose nanofibrils (CNF)) in combination with one or more inorganic minerals. The materials are combined and formed into a membrane in such a way that the resulting material exhibits rapid and controlled aqueous wicking characteristics, including, but not limited to, internal and surface wetting phenomena, internal pore volume, surface uniformity, and other improved physical properties.

In some embodiments, the present invention provides membranes well suited for use in, for example, lateral flow devices, diagnostic devices and other point of care devices (e.g., an ELISA test), auto-sampling devices (e.g., environmental testing strips), devices for concentrating biological or environment samples, devices for separating and immobilizing analytes, and as universal horizontal and vertical wicking substrates.

The present invention also provides, among other things, methods of making membranes that exhibit said unique properties. One aspect of the disclosure provides a membrane comprising a porous matrix material, wherein the porous matrix material comprises: (i) wood pulp; (ii) cellulose nanofilbrils (CNF); and (iii) one or more wetting minerals. In some embodiments, the one or more wetting mineral comprises calcium carbonate (CaCO₃), TiO₂, Alumina, Fiberglass, or a combination thereof. In some embodiments, the CNF is present in a concentration within the range of 0.1 to 1.5 wt % based on dry mass basis. In some embodiments, the one or more wetting minerals are present in a concentration within the range of 0.1 to 20 wt % of the porous matrix material. In some embodiments, the CNF comprises CNF obtained by TEMPO (2,2,6,6-tetramethylpiperidine-1-oxyl radical)-mediated oxidation.

In some embodiments, when contacted with a fluid comprising an analyte, the analyte solution travels across the membrane through capillary action. In some embodiments, the analyte is immobilized on a specific site of the membrane. In some embodiments, the analyte travels across the membrane at a rate of greater than about 0.5 mm per second. In some embodiments, the analyte is or comprises a biological material.

In some embodiments, the porous matrix material is substantially homogeneous. In some embodiments, the porous matrix material comprises a porosity of at least 60-90%. In some embodiments, the porous matrix material comprises one or more additives. In some embodiments, the one or more additives comprises a foaming agent, a blowing agent, a templating agent, a plasticizer, or a combination thereof. In some embodiments, the one or more additives are present in a concentration within the range of 0.1 to 10 wt % based on dry mass basis. In some embodiments, the foaming agent comprises a surfactant. In some embodiments, the surfactant comprises glucosides and/or myristic acid. In some embodiments, the surfactant comprises a biosurfactant such as fungi, bacteria, yeast, glycolipids, phospholipids, glycopeptides, saponins, fatty acids, proteins, polysaccharides or a combination thereof. In some embodiments, the blowing agent comprises sodium bicarbonate. In some embodiments, the templating agent comprises salt, ice, dry ice or a combination thereof. In some embodiments, the plasticizer comprises acetylated monoglycerides, alkyl citrates, epoxidized soybean oil, proteins, polyethylene glycol, fatty acids, or combinations thereof.

In another aspect, the disclosure features a method comprising: (i) providing slurry comprising wood pulp and water; (ii) mixing cellulose nanofilbrils (CNF) and one or more wetting minerals into the slurry; and (iii) drying the slurry to form a porous matrix material. In some embodiments, the one or more wetting minerals comprises calcium carbonate (CaCO₃), TiO₂, Alumina, Fiberglass, or a combination thereof. In some embodiments, drying the slurry comprises capillary dewatering, infrared drying, lyophilization, and/or microwave irradiation.

In some embodiments, the concentration of CNF is 0.1 to 1.5 wt % of the porous matrix material. In some embodiments, the one or more wetting minerals are present in a concentration within the range of 0.1 to 20 wt % of the porous matrix material.

In another aspect, the disclosure features a method of separating an analyte from a fluid comprising: (i) providing a membrane comprising a porous matrix material; and (ii) contacting the membrane with a fluid comprising an analyte so that the fluid enters the membrane through capillary action, thereby separating the analyte; wherein the porous matrix material is a composite material that comprises wood pulp, CNF, and one or more wetting minerals.

In some embodiments, the one or more wetting minerals comprises calcium carbonate (CaCO₃), TiO₂, Alumina, Fiberglass, or a combination thereof.

In some embodiments, the contacting step is or comprises contacting the membrane with a fluid contained in an adjacent space or adjacent material.

In some embodiments, the fluid travels across the membrane. In some embodiments, the fluid passively travels across the membrane. In some embodiments, the fluid travels across the membrane with the aid of a vacuum or positive pressure on the fluid. In some embodiments, the analyte is immobilized on the membrane. In some embodiments, the immobilized analyte is or comprises a biological material.

### BRIEF DESCRIPTION OF THE DRAWING

The drawings are for illustration purposes only, not for limitation.

FIG. 1 shows SEM images of a pulp membrane with 1 wt % CNF (Panel a), a pulp membrane with 5 wt % CNF (Panel b), and a CNF membrane (Panel c).

FIG. 2 shows the results of a vertical wicking test conducted on various materials including pulp membranes, CNF membranes, pulp+CNF membranes, CNF+CaCO₃ membranes, and pulp+CNF+CaCO₃ membranes. Wicking results are represented as vertical wicking time in seconds (y-axis) vs. wicking height in mm (x-axis).

FIG. 3 shows the wicking rate of various pulp-CNF—CaCO₃ membranes with varying wt % of CNF. The results are represented as wt % of CNF (x-axis) vs. vertical wicking rate in mm/sec (y-axis).

FIG. 4 is a schematic of an analyte-target interaction on an exemplary membrane.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), an oral or nasal swab, etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Biomarker: The term “biomarker” is used herein, consistent with its use in the art, to refer to a to an entity, event, or characteristic whose presence, level, degree, type, and/or form, correlates with a particular biological event or state of interest, so that it is considered to be a “marker” of that event or state. To give but a few examples, in some embodiments, a biomarker may be or comprise a marker for a particular disease state, or for likelihood that a particular disease, disorder or condition may develop, occur, or reoccur. In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. A biomarker may be or comprise an entity of any chemical class, and may be or comprise a combination of entities. For example, in some embodiments, a biomarker may be or comprise a nucleic acid, a polypeptide, a lipid, a carbohydrate, a small molecule, an inorganic agent (e.g., a metal or ion), or a combination thereof. In some embodiments, a biomarker is a cell surface marker. In some embodiments, a biomarker is intracellular. In some embodiments, a biomarker is detected outside of cells (e.g., is secreted or is otherwise generated or present outside of cells, e.g., in a body fluid such as blood, urine, tears, saliva, cerebrospinal fluid, etc. In some embodiments, a biomarker may be or comprise a genetic or epigenetic signature. In some embodiments, a biomarker may be or comprise a gene expression signature.

Cellulose Nanofibrils: As used herein, the term “cellulose nanofibrils” refers to the state of cellulosic material wherein at least 75% of the cellulosic material would be considered to be “fines”. In some embodiments, the proportion of cellulosic material that may be considered fines may be much higher such as 80%, 85%, 90%, 95%, 99% or higher. In this disclosure, the terms “nanofibrils”, “nanocellulose”, “highly fibrillated cellulose”, and “super-fibrillated cellulose” are all considered synonymous with cellulose nanofibrils.

Detectable entity: The term “detectable entity” as used herein refers to any element, molecule, functional group, compound, fragment or moiety that is detectable. In some embodiments, a detectable entity is provided or utilized alone. In some embodiments, a detectable entity is provided and/or utilized in association with (e.g., joined to) another agent. Examples of detectable entities include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes, chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

Fines: As used herein, the term “fines” refers to cellulosic material, or a portion of a cellulosic fiber with a weighted fiber length of less than 0.2 mm. In some embodiments, “fines” may refer to a cellulosic material that has a diameter of between 5 nm-100 nm, inclusive, and has a high surface to volume ratio and a high length/diameter (aspect) ratio.

Improve, increase, or reduce: As used herein, the terms “improve,” “increase” or “reduce,” or grammatical equivalents, indicate values that are relative to a baseline measurement, such as a measurement in the same sample prior to initiation of a treatment or process step described herein, or a measurement in a control sample (or multiple control samples) in the absence of a treatment or process step described herein.

Porosity: The term “porosity” as used herein, refers to a measure of void spaces in a material and is a fraction of volume of voids over the total volume, as a percentage between 0 and 100%. A determination of porosity is known to a skilled artisan using standardized techniques, for example mercury porosimetry and gas adsorption (e.g., nitrogen adsorption).

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchoalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Substantially: As used herein, the term “substantially” refers to the qualitative condition of exhibiting total or near-total extent or degree of a characteristic or property of interest. One of ordinary skill in the chemical arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### DETAILED DESCRIPTION

The present invention relates generally to the field of products made from cellulosic materials, (e.g., pulp, fiber, and nanofiber), such as membranes that exhibit rapid and controlled aqueous wicking characteristics, including, but not limited to, internal and surface wetting phenomena, internal pore volume, surface uniformity, and improved physical properties.

Nanofibrillated celluloses have previously been shown to be useful as reinforcing materials in wood and polymeric composites, as barrier coatings for paper, paperboard and other substrates, and as a papermaking additive to control porosity and bond dependent properties. A number of groups are looking at the incorporation of nanocellulose materials into paper or other products; while other research groups are looking at using this material at low concentrations for reinforcements of certain plastic composites.

Prior to the present disclosure, most wicking membranes were made from nitrocellulose, or other cellulose materials that have been significantly chemically modified (i.e., by nitration, sulfation, etc.). In addition, these nitrocellulose membranes are typically made by freeze-drying, and are limited in their wicking ability.

The present disclosure provides new membrane compositions and methods for producing membranes with improved wicking capabilities as well as other improved physical characteristics using one or more cellulosic components. These membranes can be tailored for different uses, including for biomedical applications. Additionally, these new membrane compositions, can be formed, e.g., by simpler drying methods such as oven or microwave drying, allowing for better control of the physical properties of the membranes.

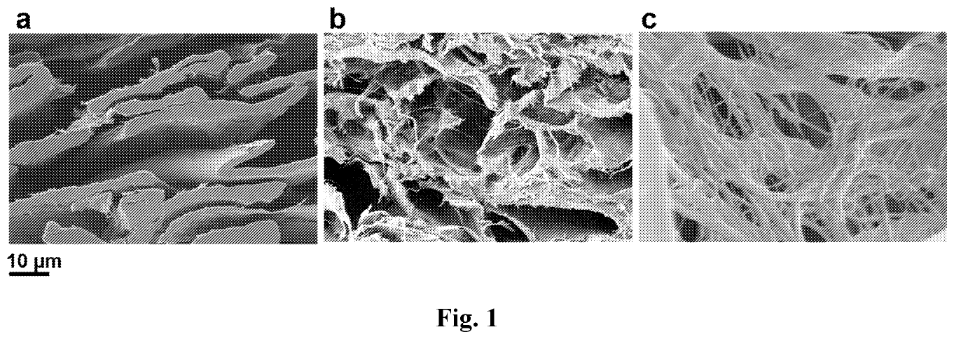

The inventors have identified that certain properties of membranes impart wicking ability. For example, materials that achieve superior wicking often include a higher porosity, and/or form lamellar-like channels (see e.g., a porous, pulp-based material shown in FIG. 1a). Further, even distribution of the each component (including additives and wetting minerals) throughout the membrane formed is desirable for e.g., in lateral flow assays and point of care/diagnostic devices.

The inventors have successfully identified compositions that include one or more cellulosic materials (e.g., wood pulp and CNF), and one or more wetting minerals, that, when formed into a membrane, exhibit superior wicking ability. The inventors have achieved a substantially homogeneous membrane material, where the wetting mineral(s) is/are distributed evenly throughout the membrane by the addition of CNF in particular quantities. The inventors have found that membranes with high CNF content (e.g., greater than about 1.5 wt % CNF) contain web-like structures (similar to CNF-based materials) and obstruct lamellar-like channels formed by pores of pulp material. In contrast, provided materials and membranes are able to achieve superior wicking properties with a lower concentration of CNF.

Further, the inventors have found that the addition of a small amount of CNF to membrane compositions improves retention of wetting minerals in the membranes. In some embodiments, CNF is added to compositions of wood pulp and wetting mineral in a concentration within the range of 0.1 to 1.5 wt % in an aqueous suspension before the membrane is dried). Surprisingly, the inventors have found that it is the specific ratios of the membrane components (pulp, CNF, and wetting mineral) that allows for both 1) the ability to retain a wetting mineral evenly throughout the membrane material; and 2) the ability to maintain a porous channel-like structure that can effectively wick liquids.

### Cellulosic Materials

According to various embodiments, any of a variety of cellulosic materials may be used in provided compositions and membranes. A cellulosic material can be any material that includes cellulose. Cellulose is found naturally in plant stems, leaves, husks, shells and cobs, or leaves, branches and xylem of trees. Cellulose materials can also be herbaceous materials, agricultural residues, forestry residues. In some embodiments, the cellulosic material is or comprises pulp fibers, microcrystalline cellulose, and cellulosic fibril aggregates. In some embodiments, a cellulosic material is or comprises a micron-scale cellulose. In some embodiments, a cellulosic material is or comprises a nano-scale cellulose (i.e. nanocellulose). In some embodiments, the nanocellulose is or comprises cellulose nanofibrils. In some embodiments, the cellulose nanofibrils are or comprise microfibrillated cellulose, nanocrystalline cellulose, and bacterial nanocellulose.

### Lignocellulosic Materials

According to various embodiments, a cellulosic material used in provided compositions and membranes is or comprises any of a variety of lignocellulosic materials. In some embodiments, a lignocellulosic material is a material comprised of and/or derived from natural polymers based on lignin, cellulose, and hemicellulose obtained from materials such as wood, wood waste, spent pulping/fractionation liquors, algal biomass, food waste, grasses, straw, corn stover, corn fiber, agricultural products and residuals, forest residuals, saw dust, wood shavings, sludges and municipal solid waste, bacterial cellulose and mixtures thereof. In some embodiments, a lignocellulosic material is or comprises wood pulp, such as chemically bleached wood pulp (soft or hardwood), and wood residues (wood flour).

In some embodiments, a micron-scale cellulose or a nano-scale cellulose is obtained from a lignocellulosic material before and/or during preparation of a provided membrane.

### Cellulose Nanofibrils (CNF)

In accordance with various embodiments, any of a variety of application-appropriate cellulose nanofibrils may be used. Nanofibrils of cellulose are also known in the literature as microfibrillated cellulose (MFC), cellulose microfibrils (CMF), nanofibrillated cellulose (NFC) and cellulose nanofibrils (CNF), but these are different from nanocrystalline cellulose (NCC) or cellulose nanocrystals (CNC). In some embodiments, CNF comprises 2,2,6,6-Tetramethylpiperidin-1-yl)oxyl (TEMPO) oxidized CNF. In some embodiments, CNF comprises lignin-containing CNF (L-CNF).

Despite the variability in nomenclature, various embodiments are applicable to nanocellulose fibers independent of the actual physical dimensions, provided at least one dimension (typically a fiber width) is in the nanometer range. CNF are generally produced from wood pulps by a refining, grinding, or homogenization process, described below, that governs the final length and length distribution. The fibers tend to have at least one dimension (e.g. diameter) in the nanometer range, although fiber lengths may vary from 0.1 μm to as much as about 4.0 mm depending on the type of wood or plant used as a source and the degree of refining. In some embodiments, the “as refined” fiber length is from about 0.2 mm to about 0.5 mm. Fiber length is measured using industry standard testers, such as the TechPap Morphi Fiber Length Analyzer. Within limits, as the fiber is more refined, the % fines increases and the fiber length decreases.

CNF or lignocellulose materials may be processed by e.g., oxidation and/or homogenization to produce specific forms of CNF materials such as 2,2,6,6-Tetramethylpiperidin-1-yl)oxyl (TEMPO) oxidized CNF.

In some embodiments, CNF are obtained from wood-based materials or residues. In some embodiments, wood-based residues comprise sawdust. In some embodiments, wood-based residues comprise wood flour. In some embodiments, wood-based residues comprise wood shavings. In some embodiments, wood-based residues comprise woodchips. These types of CNF material are normally known as lignin-containing cellulose nanofibrils (LCNF).

### Wetting Agents

In some embodiments, membranes of the present disclosure further comprise one or more wetting agents, to improve wicking of a sample on the membranes described herein.

In some embodiments, a wetting agent is an inorganic mineral or wetting mineral. A wetting mineral, e.g., can be any metal oxide that is hydrophilic in nature. Adding a wetting mineral to a cellulosic membrane imparts, in what would normally be a hydrophobic membrane, hydrophilic properties. In some embodiments, the one or more wetting agents are added into a slurry comprising one or more cellulosic materials before the membrane is formed.

Exemplary wetting minerals include any metal oxide that is hydrophilic in nature, e.g., CaCO₃, SiO₂, Alumina, hydroxyapatite, calcium phosphate, and TiO₂.

In some embodiments, one or more wetting agents may be present in a slurry or membrane in concentrations varying from about 0.01% by weight to about 80% by weight (either total weight or dry mass basis). In some embodiments, one or more wetting agents may be present in a slurry or membrane in concentrations varying from about 0.01% by weight to about 20% by weight (either total weight or dry mass basis). In some embodiments, one or more wetting agents may be present in a slurry or membrane in concentrations varying from about 0.01-75%, 0.01-70%, 0.01-65%, 0.01-60%, 0.01-55%, 0.01-50%, 0.01-45%, 0.01-40%, 0.01-35%, 0.01-30%, 0.01-25%, 0.01-20%, 0.01-15%, 0.01-10%, 0.01-5%, 0.01-1%, 0.01-0.5% by weight (either total weight or dry mass basis).

### Additives

In some embodiments, membranes of the present disclosure comprise one or more additives. In some embodiments, an additive is added to a slurry comprising one or more cellulosic materials, which is formed into a membrane.

In some embodiments, one or more additives modify physical, mechanical or chemical properties of a membrane relative to an identical membrane lacking the one or more additives. In some embodiments, one or more additives comprise wood derivatives, metal particles, latex particles, bioceramics, glass materials, proteins, fluorescent dyes, minerals, natural fibers, polymer materials, or any combination thereof. In some embodiments, an additive is or comprises wood derivatives.

In some embodiments, an additive comprises one or more foaming agents, blowing agents, and/or templating agents. Foaming agents include e.g., synthetic surfactants (both ionic and nonionic), biosurfactants such fungi, bacteria, yeast, glycolipids, phospholipids, glycopeptides, saponins, fatty acids, proteins, polysaccharides. Blowing agent include e.g., CO₂, baking soda, etc. A templating agent includes e.g., a salt, ice, dry ice.

In some embodiments, an additive comprises one or more plasticizers. Example plasticizer agents include, for example, acetylated monoglycerides, alkyl citrates, epoxidized soybean oil, proteins, PEGS, fatty acids, etc. or surfactants (glucosides (coco, decyl, lauryl, etc.), and myristic acid, etc.).

In some embodiments, an additive comprises one or more flame retardants. Example flame retardants include, for example, carbon (graphite, graphene, nanotubes), brominated polymer, chlorinated anhydrides, acids and paraffins, minerals (clay, borates, hydroxides of aluminum and magnesium, zinc stannates), nitrogen (melamines), phosphorous (red phosphorous, organophosphates, halogenated phosphates, ammonium polyphosphates, phosphine oxides), silicon-based additives, organic acids, and carbonates.

In some embodiments, an additive is or comprises metal particles. In some embodiments, an additive is or comprises metal oxide particles. In some embodiments, metal particles are silver particles. In some embodiments, metal particles are gold particles. In some embodiments, metal oxide particles are titanium oxide particles. In some embodiments, metal oxide particles are iron oxide particles. In some embodiments, metal oxide particles are silver dioxide particles. In some embodiments, metal oxide particles are aluminum oxide particles.

In some embodiments, an additive is or comprises a stabilizing agent. An example stabilizing agent includes citric acid.

In some embodiments, an additive is or comprises latex particles.

In some embodiments, an additive is or comprises one or more bioceramic materials. In some embodiments, a bioceramic material is or comprises one or more of tricalcium phosphate, a tricalcium phosphate derivative, dicalcium phosphate, a dicalcium phosphate derivative, or any combination thereof.

In some embodiments, an additive is or comprises one or more glass materials. In some embodiments, glass materials are bioactive. In some embodiments, glass materials comprise glass fibers, glass beads, glass particles, or any combination thereof.

In some embodiments, an additive is or comprises one or more proteins. In some embodiments, proteins comprise growth factors.

In some embodiments, an additive is or comprises one or more fluorescent dyes. In some embodiments, a fluorescent dye comprises one or more fluorescent tags.

In some embodiments, an additive comprises one or more minerals. In some embodiments, a mineral may be or comprise hydroxyapatite, hydroxyapatite derivatives, cement, concrete, clay, or any combination thereof.

In some embodiments, an additive comprises one or more natural fibers. In some embodiments, an additive comprises polymer fibers.

Other additives are known to those skilled in the art and could be considered for addition to the structural products of the invention without deviating from the scope of the invention.

In some embodiments, one or more additives may be present in concentrations varying from about 0.01% by weight to about 80% by weight. In some embodiments, one or more additives may be present in concentrations varying from about 0.01-75%, 0.01-70%, 0.01-65%, 0.01-60%, 0.01-55%, 0.01-50%, 0.01-45%, 0.01-40%, 0.01-35%, 0.01-30%, 0.01-25%, 0.01-20%, 0.01-15%, 0.01-10%, 0.01-5%, 0.01-1%, 0.01-0.5%, 0.01-0.1%, 0.01-0.09%, 0.01-0.08%, 0.01-0.07%, 0.01-0.06%, 0.01-0.05%, 0.01-0.04%, 0.01-0.03%, or 0.01-0.02% by weight. In some embodiments, one or more additives may be present in concentrations varying from about 0.05-80%, 0.1-80%, 0.5-80%, 1-80%, 5-80%, 10-80%, 15-80%, 20-80%, 25-80%, 30-80%, 35-80%, 40-80%, 45-80%, 50-80%, 55-80%, 60-80%, 65-80%, 70-80%, 71-80%, 72-80%, 73-80%, 74-80%, 75-80%, 76-80%, 77-80%, 78-80%, or 79-80% by weight.

An exemplary additive that imparts a change in a chemical property of a composition is the addition of a reagent to a cellulosic structure. Reagents in diagnostic applications may include analyte capture reagents such as antibodies or fragments thereof. Reagents in environmental applications may include any chemical reagents known to react with and detect the presence of an environmental contaminant or other analyte. Through the control of disintegration characteristics and porosity, the reagents may be gradually released into the surroundings.

### General Pulping and CNF Processes

Pulp used in making provided membranes and compositions can be obtained by any known pulping process. Example chemical pulping processes include: (a) the Kraft process, (b) the sulfite process, and (c) the soda process, and these are well described in the literature, in e.g., Smook, Gary A., Handbook for Pulp & Paper Technologists, Tappi Press, 1992 (e.g., in Chapter 4), and the article: “Overview of the Wood Pulp Industry,” Market Pulp Association, 2007.

CNF used in making provided membranes and compositions can be obtained via any known process for producing nanocellulose or fibrillated cellulose, for example, those processes disclosed in U.S. Pat. No. 10,563,352, which is herein incorporated by reference in its entirety. In some embodiments, the process of obtaining CNF includes a step in which the wood pulp is mechanically comminuted in any type of mill or device that grinds the fibers apart. Such mills are known in the art and include, without limitation, Valley beaters, single disk refiners, double disk refiners, conical refiners, including both wide angle and narrow angle, cylindrical refiners, homogenizers, microfluidizers, and other similar milling or grinding apparatus. Example mechanical comminution devices can be found, for example, in Smook, Gary A., Handbook for Pulp & Paper Technologists, Tappi Press, 1992 (e.g., in Chapter 13). The process of mechanical breakdown or comminution, regardless of instrument type, is sometimes referred to in the pulp literature as “refining.”

The extent of refining may be monitored during the process by any of several means. Certain optical instruments can provide continuous data relating to the fiber length distributions and percent fines, either of which may be used to define endpoints for the comminution stage. Within limits, as the fiber is more refined, the % fines increases and the fiber length decreases. Fiber length is measured using industry standard testers, such as the TechPap Morphi Fiber Length Analyzer, which reads out a particular “average” fiber length. In some embodiments, the “as refined” fiber length is from about 0.1 mm to about 0.6 mm, or from about 0.2 mm to about 0.5 mm.

In some embodiments, prior to refining (e.g., by homogenization), pulp may be chemically modified (e.g., by sulfation or nitration).

Refining pulp to produce highly fibrillated cellulose may be done using various mechanical treatments, such as by using homogenizers and/or ultrafine grinders. In some embodiments, a low consistency refiner may be used to produce CNF, as described, e.g., in U.S. Pat. No. 7,381,294 (Suzuki et al.). In some embodiments, microfibrillated cellulose or CNF can be produced by recirculating fiber slurry through a refiner. In some embodiments, two refiners are used sequentially.

By way of non-limiting example, U.S. Pat. No. 9,988,762 describes a refining process for preparing CNF from wood products, and is incorporated herein in its entirety. In some embodiments, the process of obtaining CNF involves processing a slurry of cellulosic fibers, preferably wood fibers, which have been liberated from the lignocellulosic matrix using a pulping process. The pulping process can be a chemical pulping process such as the sulfate (e.g., Kraft) or sulfite process. The process may include first and second mechanical refiners which apply shear to the fibers. The refiners can be low consistency refiners. In such embodiments, shear forces help to break up the fiber's cell walls, exposing the fibrils and nanofibrils contained in the wall structure. Mechanical treatment may continue until the desired quantity of fibrils is liberated from the fibers.

In certain refining processes, a large volume of water is employed. As noted, the slurries may comprise 90-99% (by weight) of water and only 1-10% fibers. If desired, complete water removal is routinely achieved through conventional means (evaporation, freeze-drying, electrospraying, oven heating, microwave, etc.) and/or combined with other materials in order to achieve a particular final form (e.g., a membrane material).

### Methods of Forming Membranes

The present disclosure provides, among other things, methods of making a membrane comprising one or more cellulosic components, wherein the one or more cellulosic components comprise a micron-scale cellulose or cellulose nanofibrils (CNF), wood pulp, and wetting agent(s), the method comprising the steps of (i) creating a cellulosic slurry by combining the one or more of cellulosic components and wetting agent(s) with a liquid component; (ii) mixing the components of the cellulosic slurry and (iii) exposing the cellulosic slurry to a drying condition, thereby forming a membrane.

### Cellulosic Slurry

In accordance with various embodiments of the present invention, cellulosic slurries are used in compositions for making membranes. In some embodiments, cellulosic slurries comprise one or more cellulosic materials suspended in a liquid component, such as water. In some embodiments, a slurry comprises a suspension, colloid, mixture, emulsion, or hydrogel. In some embodiments, a cellulosic component comprises a micron-scale cellulose. In some embodiments, a cellulosic component comprises CNF. In some embodiments, a cellulosic component comprises wood-based residues.

In some embodiments, a cellulosic slurry comprises wood and/or other lignocellulosic derivatives. In some embodiments, wood derivatives may be or comprise wood flour, wood pulp, or a combination thereof.

In some embodiments, a cellulosic slurry comprises about 0.01 wt % to about 10 wt % (e.g., 0.01 to 0.1 wt %, 0.1 to 1.5 wt %, 0.1 to 2 wt %, 0.1 to 5 wt %, 1 to 10 wt %) CNF by dry mass basis, wherein the wt % is calculated based on the total weight of all solid components present in the slurry (and excludes the weight of the liquid components).

In some embodiments, a cellulosic slurry comprises about 0.01 wt % to about 10 wt % (e.g., 0.01 to 0.1 wt %, 0.1 to 1.5 wt %, 0.1 to 2 wt %, 0.1 to 5 wt %, 1 to 10 wt %) of pulp (e.g., soft and/or hard wood pulp), wherein the wt % is calculated based on the dry mass, wherein the wt % is calculated based on the total weight of all solid components present in the slurry (and excludes the weight of the liquid components).

In some embodiments, a cellulosic slurry comprises CNF and a wetting agent (e.g., a wetting mineral). In some embodiments, the ratio of CNF:wetting mineral present in a cellulosic slurry is within a range of about 1:0.0001 to about 1:1000. In some embodiments, the ratio of CNF:wetting mineral present in a cellulosic slurry is within a range of about 1:0.0001-0.001, 1:0.001-0.1, 1:0.1-1, 1:1-5, 1:5-10, 1:10-20, 1:20-50, 1:50-100, or about 1:100-1000. In some embodiments, the ratio of CNF:wetting mineral present in a cellulosic slurry is about 1:0.0001, 1:001, 1:0.01, 1:0.1, 1:0.2, 1:0.3, 1:0.4, 1:0.5, 1:0.6, 1:0.7, 1:0.8, 1:0.9, 1:1, 1:2, 1:3, 1:4, 1:5, 1:10, 1:12, 1:14, 1:15, 1:20, 1:50, 1:100 or about 1:1000.

In some embodiments, a wetting agent is added to dry CNF and subsequently combined with water to form a suspension.

In some embodiments, a cellulosic slurry comprises pulp and CNF. In some embodiments, the ratio of pulp:CNF present in a cellulosic slurry is within a range of about 1:0.0001 to about 1:1000. In some embodiments, the ratio of pulp:CNF present in a cellulosic slurry is within a range of about 1:0.0001-0.001, 1:0.001-0.1, 1:0.1-1, 1:1-5, 1:5-10, 1:10-20, 1:20-50, 1:50-100, or about 1:100-1000. In some embodiments, the ratio of pulp:CNF present in a cellulosic slurry is about 1:0.0001, 1:001, 1:0.01, 1:0.1, 1:0.2, 1:0.3, 1:0.4, 1:0.5, 1:0.6, 1:0.7, 1:0.8, 1:0.9, 1:1, 1:2, 1:3, 1:4, 1:5, 1:10, 1:12, 1:14, 1:15, 1:20, 1:50, 1:100 or about 1:1000.

In some embodiments, a cellulosic slurry comprises an additive. In some embodiments, a cellulosic slurry comprises 0.01-95 wt % of additive(s), wherein the wt % is calculated based on the dry mass, wherein the wt % is calculated based on the total weight of all solid components present in the slurry (and excludes the weight of the liquid components). For example, in some embodiments, a cellulosic slurry may comprise between 0.01% and 95% (e.g., between 0.01 and 90%, 0.01 and 80%, 0.01 and 70%, 0.01 and 60%, 0.01 and 50%, 0.01 and 40%, 0.01 and 30%, 0.01 and 20%, 0.01 and 10%, or 0.01 and 5%) wt % additive(s). In some embodiments, a cellulosic slurry comprises at least 0.01 wt % additive(s) (e.g., at least 0.01 wt %, 0.1 wt %, 0.5 wt %. 1 wt %, 5 wt %, 10 wt %, 15 wt %, 20 wt %).

In some embodiments, the ratio of another solid component (e.g., a cellulosic material such as pulp and/or CNF): additive present in the cellulosic slurry is within a range of about 1:0.0001 to about 1:1000. In some embodiments, the ratio of another solid component (e.g., a cellulosic material such as pulp and/or CNF): additive present in the cellulosic slurry is within a range of about 1:0.001 to about 1:100. In some embodiments, the ratio of another solid component (e.g., a cellulosic material such as pulp and/or CNF): additive present in the cellulosic slurry is about 1:0.0001, 1:0.001, 1:0.01, 1:0.1, 1:0.2, 1:0.3, 1:0.4, 1:0.5, 1:0.6, 1:0.7, 1:0.8, 1:0.9, 1:1, 1:2, 1:3, 1:4, 1:5, 1:10, 1:12, 1:14, 1:15, 1:20, 1:50, 1:100 or about 1:1000. In some embodiments, the ratio of another solid component (e.g., a cellulosic material such as pulp and/or CNF): additive present in a cellulosic slurry is within a range of about 1:0.0001-0.001, 1:0.001-0.1, 1:0.1-1, 1:1-5, 1:5-10, 1:10-20, 1:20-50, 1:50-100, or about 1:100-1000.

In some embodiments, the total solid content of the cellulosic slurry is in the range of 0.01-10 wt % (e.g., 0.01 to 0.1 wt %, 0.1 to 1.5 wt %, 0.1 to 2 wt %, 0.1 to 5 wt %, 1 to 10 wt %), wherein the wt % is calculated based on the dry mass, which is calculated based on the total weight of all solid components present in the slurry (and excludes the weight of the liquid components).

In some embodiments, a cellulosic slurry comprises a liquid component wherein the liquid component is water. In some embodiments, a cellulosic slurry comprises a liquid component wherein the liquid component is an alcohol. In some embodiments, an alcohol is ethanol. In some embodiments, a liquid component comprises a mixture of water and an alcohol. In some embodiments, a liquid component is acetone.

In some embodiments, pulp is soaked in a liquid (e.g., DI water) for a period of time before it is further processed. In some embodiments, pulp is soaked in a liquid for a period of time ranging from 1 hour to 7 days, e.g., 24 hours. In some embodiments, pulp is soaked in a liquid for a period of time that is at least e.g., 1, 2, 3, 4, 8, 16, 24 hours, 48 hours, 72 hours or more.

### Mixing

Membranes of the present disclosure can be formed, for example, by providing a slurry comprising one or more cellulosic materials, a wetting agent, and a liquid, and mixing the components of the slurry to distribute and combine the components.

In some embodiments, a slurry is prepared via mechanical mixing. In some embodiments, mixing is accomplished via automated mixing, e.g., using equipment such as automated disintegrator, blender, automated shaker, ultra sonicator or a planetary mixer.

In some embodiments, a pulp slurry and a slurry containing CNF and wetting mineral suspensions are mixed, using any application-appropriate method, for example, mixing techniques such as mechanical mixing. In some embodiments, mixing is accomplished via automated mixing, e.g., using equipment such as automated disintegrator, blender, automated shaker, ultra sonicator or a planetary mixer.

In some embodiments, mixing components of a cellulosic slurry comprises one or more mixing sessions. In some embodiments, one or more mixing sessions (e.g., three mixing sessions) are separated in time by intervals ranging from minutes to days (e.g., at least one minute, 5 minutes, 10 minutes, 20 minutes, 30 minutes, 40 minutes, 50 minutes, one hour, two hours, 24 hours, 40 hours or more).

In some embodiments, a first mixing session is used to mix CNF with an additive (e.g., a wetting mineral), forming a first mixture. In some embodiments, a second mixing session is used to mix pulp with water, forming a second mixture. In some embodiments, another mixing session is used to mix the first mixture and the second mixture. In some embodiments, each mixing session is about 30 minutes.

In some embodiments, one or more mixing sessions comprise identical mixing conditions. In some embodiments, one or more mixing sessions comprise conditions that vary in one or more parameters (e.g., time, intensity, volume of material, type of mixing/device used for mixing) from at least one other mixing session.

In some embodiments, a cellulosic slurry is mixed for a duration comprising about 10 seconds to about 3 hours. In some embodiments, a cellulosic slurry is mixed for a duration comprising about 10 seconds to about 2 hours. In some embodiments, a cellulosic slurry is mixed for a duration comprising about 10 seconds to about 1 hour. In some embodiments, a cellulosic slurry is mixed for a duration comprising about 10 seconds to about 55 minutes. In some embodiments, a cellulosic slurry is mixed for a duration comprising about 10 seconds to about 50 minutes. In some embodiments, a cellulosic slurry is mixed for a duration comprising about 10 seconds to about 45 minutes. In some embodiments, a cellulosic slurry is mixed for a duration comprising about 10 seconds to about 40 minutes. In some embodiments, a cellulosic slurry is mixed for a duration comprising about 10 seconds to about 35 minutes. In some embodiments, a cellulosic slurry is mixed for a duration comprising about 10 seconds to about 30 minutes. In some embodiments, a cellulosic slurry is mixed for a duration comprising about 10 seconds to about 25 minutes. In some embodiments, a cellulosic slurry is mixed for a duration comprising about 10 seconds to about 20 minutes. In some embodiments, a cellulosic slurry is mixed for a duration comprising about 10 seconds to about 15 minutes. In some embodiments, a cellulosic slurry is mixed for a duration comprising about 10 seconds to about 10 minutes. In some embodiments, a cellulosic slurry is mixed for a duration comprising about 10 seconds to about 9 minutes. In some embodiments, a cellulosic slurry is mixed for a duration comprising about 10 seconds to about 8 minutes. In some embodiments, a cellulosic slurry is mixed for a duration comprising about 10 seconds to about 7 minutes. In some embodiments, a cellulosic slurry is mixed for a duration comprising about 10 seconds to about 6 minutes. In some embodiments, a cellulosic slurry is mixed for a duration comprising about 10 seconds to about 5 minutes. In some embodiments, a cellulosic slurry is mixed for a duration comprising about 10 seconds to about 4 minutes. In some embodiments, a cellulosic slurry is mixed for a duration comprising about 10 seconds to about 3 minutes. In some embodiments, a cellulosic slurry is mixed for a duration comprising about 10 seconds to about 2 minutes. In some embodiments, a cellulosic slurry is mixed for a duration comprising about 10 seconds to about 1 minute. In some embodiments, a cellulosic slurry is mixed for a duration comprising about 10 seconds to about 55 seconds. In some embodiments, a cellulosic slurry is mixed for a duration comprising about 10 seconds to about 50 seconds. In some embodiments, a cellulosic slurry is mixed for a duration comprising about 10 seconds to about 45 seconds. In some embodiments, a cellulosic slurry is mixed for a duration comprising about 10 seconds to about 40 seconds. In some embodiments, a cellulosic slurry is mixed for a duration comprising about 10 seconds to about 35 seconds. In some embodiments, a cellulosic slurry is mixed for a duration comprising about 10 seconds to about 30 seconds. In some embodiments, a cellulosic slurry is mixed for a duration comprising about 10 seconds to about 25 seconds. In some embodiments, a cellulosic slurry is mixed for a duration comprising about 10 seconds to about 20 seconds. In some embodiments, a cellulosic slurry is mixed for a duration comprising about 10 seconds to about 19 seconds. In some embodiments, a cellulosic slurry is mixed for a duration comprising about 10 seconds to about 18 seconds. In some embodiments, a cellulosic slurry is mixed for a duration comprising about 10 seconds to about 17 seconds. In some embodiments, a cellulosic slurry is mixed for a duration comprising about 10 seconds to about 16 seconds. In some embodiments, a cellulosic slurry is mixed for a duration comprising about 10 seconds to about 15 seconds. In some embodiments, a cellulosic slurry is mixed for a duration comprising about 10 seconds to about 14 seconds. In some embodiments, a cellulosic slurry is mixed for a duration comprising about 10 seconds to about 13 seconds. In some embodiments, a cellulosic slurry is mixed for a duration comprising about 10 seconds to about 12 seconds. In some embodiments, a cellulosic slurry is mixed for a duration comprising about 10 seconds to about 11 seconds.

### Drying

Membranes of the present disclosure can be formed, for example by providing a slurry comprising one or more cellulosic materials, wetting agent(s), and a liquid, mixing the components of the slurry to distribute and combine the components, and drying the cellulosic slurry using techniques such as capillary dewatering, gravity and vacuum filtration, electric oven, infrared heating, microwave heating, freeze drying, ambient heating, or a combination thereof.

In some embodiments, a drying condition comprises one or more drying sessions. In some embodiments, one or more drying sessions are separated in time by intervals ranging from minutes to days (e.g., at least one minute, 5 minutes, 10 minutes, 20 minutes, 30 minutes, 40 minutes, 50 minutes, one hour, two hours, 24 hours, 40 hours or more).

In some embodiments, one or more drying sessions comprise identical drying conditions. In some embodiments, one or more drying sessions comprise conditions that vary in one or more parameters (e.g., time, intensity, volume of material) from at least one other drying session.

In some embodiments, a process of exposing a cellulosic slurry to one or more drying conditions can achieve a membrane comprising at least 80% cellulosic solids by weight (e.g., at least 85%, 90%, 95%, 99% cellulosic solids by weight). In some embodiments, a process of exposing a cellulosic slurry to one or more drying conditions can achieve a membrane comprising about 95% cellulosic solids by weight.

In some embodiments a first drying condition can be applied to a lignocellulosic slurry to achieve a hydrogel of about 1-60% solid content (e.g., about 1-10%, 10-20%, 20-30%, 30-40%, 40-50%, or about 50-60% solid content). In some embodiments, a second drying condition can be applied to the hydrogel for complete or near complete dehydration (e.g., a membrane of at least 90% solid content).

In some embodiments the first drying condition includes capillary dewatering, gravity and vacuum filtration, electric oven, infrared heating, microwave heating, freeze drying, and/or ambient heating.

In some embodiments the second drying condition includes capillary dewatering, gravity and vacuum filtration, electric oven, infrared heating, microwave heating, freeze drying, and/or ambient heating.

### Capillary Action

In accordance with various embodiments, provided methods include the use of capillary action to dewater a slurry containing cellulosic materials (e.g., CNF and pulp) by contacting the slurry with a surface of a porous dewatering material, and removing at least a portion of the water in the aqueous suspension via capillary action thereby forming a porous nanocellulose material. In some embodiments, the removing step continues for at least 8 hours.

In some embodiments, capillary dewatering of a cellulosic slurry may be done, for example, by placing the suspension in a porous vessel and balancing the effects of capillary pressure, hydrostatic pressure and enthalpy.

In accordance with any of a variety of embodiments, any application-appropriate porous dewatering material may be used. In some embodiments, in order to be useful in accordance with provided methods, the porous dewatering material must be able to facilitate the movement of water out of the aqueous suspension and across the porous dewatering material, for example, to an exterior surface (i.e., a surface not in contact with the aqueous suspension). In some embodiments, the porous dewatering material comprises a hydrophilic surface. In some embodiments, the porous dewatering material is selected from the group consisting of firebrick, kiln brick, cinderblock, terra cotta ceramics, and porous gypsum based materials (e.g., plaster of Paris). In some embodiments, a porous dewatering material is used in combination with a rigid material (e.g., a rigid metal material) so that the cellulosic material is simultaneously dried and shaped to the surface of the rigid material.

In some embodiments, capillary dewatering methods further include the step of controlling at least one of pressure and temperature to control a rate of water removal from a second surface of the porous dewatering material in order to control porosity of the formed membrane.

Without wishing to be bound by a particular theory, it is likely that the gentle and controlled nature of capillary forces allow for production of provided materials, as opposed to the much harsher methods used previously (e.g., hot press molding, etc) in an attempt to achieve higher proportions of solids, for example, in solution.

In some embodiments, the pressure and/or temperature is manipulated. In some embodiments, modulating the temperature comprises raising the temperature. In some embodiments, modulating the temperature comprises lowering the temperature. In some embodiments, modulating the pressure comprises increasing the pressure. In some embodiments, modulating the pressure comprises lowering the pressure. In some embodiments, controlling the pressure comprises creating at least a partial vacuum.

In some embodiments, a slurry is partially dewatered via capillary action to form a partially dried membrane, and is then further dried using other methods. In some embodiments, the remaining water is frozen in the partially dried membrane, and then further dried by evaporating the frozen remaining water from the membrane.

In some embodiments, capillary dewatering is performed using a mold, as described in U.S. patent application Ser. No. 16/086,988 (published as US Publication No.: US 2019/0093288 A1), and herein incorporated by reference in its entirety.

### Microwave Radiation

In some embodiments, a drying condition comprises microwave radiation. In some embodiments, one or more drying sessions comprise identical microwave conditions. In some embodiments, one or more drying sessions comprise microwave conditions that vary in one or more microwave parameters from at least one other drying session. In some embodiments, one or more microwave parameters comprise microwave power, microwave wavelength, microwave frequency, microwave directionality, microwave flux and duration of microwave exposure. In some embodiments, one or more drying sessions comprises one drying session and, during the one drying session, microwave radiation varies in one or more of power, wavelength, frequency, directionality and flux.

In some embodiments, microwave radiation has a power of about 5 W/kg of cellulosic slurry to about 100 kW/kg of cellulosic slurry. In some embodiments, microwave radiation has a power of about 5-90,000, 5-80,000, 5-70,000, 5-60,000, 5-50,000, 5-40,000, 5-30,000, 5-20,000, 5-10,000, 5-9,000, 5-8,000, 5-7,000, 5-6,000, 5-5,000, 5-4,000, 5-3,000, 5-2,000, 5-1,000, 5-900, 5-800, 5-700, 5-600, 5-500, 5-400, 5-300, 5-200, 5-100, 5-95, 5-90, 5-85, 5-80, 5-75, 5-70, 5-65, 5-60, 5-55, 5-50, 5-45, 5-40, 5-35, 5-30, 5-25, 5-20, 5-19, 5-18, 5-17, 5-16, 5-15, 5-14, 5-13, 5-12, 5-11, 5-10, 5-9, 5-8, 5-7, or 5-6 W/kg.

In some embodiments, microwave radiation has a wavelength of about one millimeter to about one meter. In some embodiments, microwave radiation has a wavelength of about 1-900, 1-850, 1-800, 1-750, 1-700, 1-650, 1-600, 1-550, 1-500, 1-450, 1-400, 1-350, 1-300, 1-250, 1-200, 1-150, 1-100, 1-90, 1-85, 1-80, 1-75, 1-70, 1-65, 1-60, 1-55, 1-50, 1-45, 1-40, 1-35, 1-30, 1-25, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 millimeters. In some embodiments, microwave radiation has a wavelength of about 0.005-1, 0.01-1, 0.015-1, 0.02-1, 0.025-1, 0.03-1, 0.035-1, 0.04-1, 0.045-1, 0.05-1, 0.055-1, 0.06-1, 0.065-1, 0.07-1, 0.075-1, 0.08-1, 0.085-1, 0.09-1, 0.095-1, 0.1-1, 0.2-1, 0.25-1, 0.3-1, 0.35-1, 0.4-1, 0.45-1, 0.5-1, 0.55-1, 0.6-1, 0.65-1, 0.7-1, 0.75-1, 0.8-1, 0.85-1, or 0.9-1 meters.

In some embodiments, microwave radiation may have a frequency between 500 MHz and 100 GHz, between 500 MHz and 50 GHz, between 500 MHz and 10 GHz, or between 500 MHz and 5 GHz. In some embodiments, microwave radiation may have a frequency of 915 MHz. In some embodiments, microwave radiation may have a frequency of 2,450 MHz. In some embodiments, microwave radiation may have a frequency between 915 MHz and 2,450 MHz.

In some embodiments, a cellulosic slurry is exposed to microwave radiation for a duration comprising about 10 seconds to about 3 hours. In some embodiments, a cellulosic slurry is exposed to microwave radiation for a duration comprising about 10 seconds to about 2 hours. In some embodiments, a cellulosic slurry is exposed to microwave radiation for a duration comprising about 10 seconds to about 1 hour. In some embodiments, a cellulosic slurry is exposed to microwave radiation for a duration comprising about 10 seconds to about 55 minutes. In some embodiments, a cellulosic slurry is exposed to microwave radiation for a duration comprising about 10 seconds to about 50 minutes. In some embodiments, a cellulosic slurry is exposed to microwave radiation for a duration comprising about 10 seconds to about 45 minutes. In some embodiments, a cellulosic slurry is exposed to microwave radiation for a duration comprising about 10 seconds to about 40 minutes. In some embodiments, a cellulosic slurry is exposed to microwave radiation for a duration comprising about 10 seconds to about 35 minutes. In some embodiments, a cellulosic slurry is exposed to microwave radiation for a duration comprising about 10 seconds to about 30 minutes. In some embodiments, a cellulosic slurry is exposed to microwave radiation for a duration comprising about 10 seconds to about 25 minutes. In some embodiments, a cellulosic slurry is exposed to microwave radiation for a duration comprising about 10 seconds to about 20 minutes. In some embodiments, a cellulosic slurry is exposed to microwave radiation for a duration comprising about 10 seconds to about 15 minutes. In some embodiments, a cellulosic slurry is exposed to microwave radiation for a duration comprising about 10 seconds to about 10 minutes. In some embodiments, a cellulosic slurry is exposed to microwave radiation for a duration comprising about 10 seconds to about 9 minutes. In some embodiments, a cellulosic slurry is exposed to microwave radiation for a duration comprising about 10 seconds to about 8 minutes. In some embodiments, a cellulosic slurry is exposed to microwave radiation for a duration comprising about 10 seconds to about 7 minutes. In some embodiments, a cellulosic slurry is exposed to microwave radiation for a duration comprising about 10 seconds to about 6 minutes. In some embodiments, a cellulosic slurry is exposed to microwave radiation for a duration comprising about 10 seconds to about 5 minutes. In some embodiments, a cellulosic slurry is exposed to microwave radiation for a duration comprising about 10 seconds to about 4 minutes. In some embodiments, a cellulosic slurry is exposed to microwave radiation for a duration comprising about 10 seconds to about 3 minutes. In some embodiments, a cellulosic slurry is exposed to microwave radiation for a duration comprising about 10 seconds to about 2 minutes. In some embodiments, a cellulosic slurry is exposed to microwave radiation for a duration comprising about 10 seconds to about 1 minute. In some embodiments, a cellulosic slurry is exposed to microwave radiation for a duration comprising about 10 seconds to about 55 seconds. In some embodiments, a cellulosic slurry is exposed to microwave radiation for a duration comprising about 10 seconds to about 50 seconds. In some embodiments, a cellulosic slurry is exposed to microwave radiation for a duration comprising about 10 seconds to about 45 seconds. In some embodiments, a cellulosic slurry is exposed to microwave radiation for a duration comprising about 10 seconds to about 40 seconds. In some embodiments, a cellulosic slurry is exposed to microwave radiation for a duration comprising about 10 seconds to about 35 seconds. In some embodiments, a cellulosic slurry is exposed to microwave radiation for a duration comprising about 10 seconds to about 30 seconds. In some embodiments, a cellulosic slurry is exposed to microwave radiation for a duration comprising about 10 seconds to about 25 seconds. In some embodiments, a cellulosic slurry is exposed to microwave radiation for a duration comprising about 10 seconds to about 20 seconds. In some embodiments, a cellulosic slurry is exposed to microwave radiation for a duration comprising about 10 seconds to about 19 seconds. In some embodiments, a cellulosic slurry is exposed to microwave radiation for a duration comprising about 10 seconds to about 18 seconds. In some embodiments, a cellulosic slurry is exposed to microwave radiation for a duration comprising about 10 seconds to about 17 seconds. In some embodiments, a cellulosic slurry is exposed to microwave radiation for a duration comprising about 10 seconds to about 16 seconds. In some embodiments, a cellulosic slurry is exposed to microwave radiation for a duration comprising about 10 seconds to about 15 seconds. In some embodiments, a cellulosic slurry is exposed to microwave radiation for a duration comprising about 10 seconds to about 14 seconds. In some embodiments, a cellulosic slurry is exposed to microwave radiation for a duration comprising about 10 seconds to about 13 seconds. In some embodiments, a cellulosic slurry is exposed to microwave radiation for a duration comprising about 10 seconds to about 12 seconds. In some embodiments, a cellulosic slurry is exposed to microwave radiation for a duration comprising about 10 seconds to about 11 seconds.

In some embodiments, a cellulosic slurry is contained in a mold when exposed to microwave radiation for at least one drying session (e.g., microwave radiation session). In some embodiments, a cellulosic slurry is not contained in a mold when exposed to the microwave radiation for at least one drying session.

In some embodiments, the cellulosic slurry is exposed to the microwave radiation until the liquid component content is between about 0.01% to about 20% by weight (e.g., between 0.05 to 20%, 0.05 to 10%, 0.1 to 20%, 0.1 to 10%, 1 to 20%, 1 to 15%, 1 to 10%, 1 to 5% by weight).

In some embodiments, variation in microwave radiation results in a cellulosic composition having homogenous internal void space per volume. In some embodiments, variation in microwave radiation results in a cellulosic composition having homogenous porosity.

A cellulosic slurry of the present disclosure can be dried according to any methods known in the art and is not limited to the specific examples provided herein.

### Molding

In some embodiments, a cellulosic slurry is extruded after at least one drying session. In some embodiments, a mold is cylindrical. In some embodiments, a mold is a sphere, cone, cube, sheet or thin film. In some embodiments, the shape of a membrane may be modified or altered relative to the shape of mold if, between a first and a second drying condition, a semi-solid composition is removed from a mold while it is still somewhat malleable (e.g., up to about 80% water by weight). In some embodiments, a semi-solid composition may be shaped into a non-mold shape before the composition is dried to completion in a subsequent drying condition. In some embodiments, a semi-solid composition can be shaped into a form and then exposed, mold-free, to a drying condition to obtain a desired shape.

In some embodiments, a membrane that is dry or partially dried can be attached, associated, and/or combined with another membrane (e.g., of the same or different material). In some embodiments, a membrane can be combined with another material that is chosen based on the final use of the membrane. For example, a membrane can be combined with a flexible backing for use in e.g., a lateral flow assay.

Example materials to be used in combination with provided membranes include polyethylene terephthalate (PET) fibers, such as Dacron® fibers, nitrocellulose, polyester, nylon, cellulose acetate, hydrogel, polypropylene, glass fibers, etc. . . . In some embodiments, a membrane may be combined with one or more other materials and then molded to form a final membrane material.

In some embodiments, a membrane comprises one or more layers of the same membrane material (e.g., a membrane made from the same amount of cellulosic material and wetting mineral).

In some embodiments, a membrane comprises a first layer of membrane material (e.g., a membrane made from one or more cellulosic material(s) and wetting mineral(s)) and one or more additional layers comprising a different membrane material.

### Physical Properties

The present disclosure provides membranes comprising various physical properties. The present disclosure recognizes that including specific amounts of CNF in the membranes improves dispersibility of wood pulp in water. Additionally, CNF enhances mechanical properties of membranes and retention of wetting minerals in the membranes.

In some embodiments, a membrane has a density of about 0.01 g/cm³ to about 2.5 g/cm³, e.g., about 0.01 g/cm³ to about 1.0 g/cm³. In some embodiments, a membrane has a density between about 0.02-2.4, 0.02-2.3, 0.02-2.2, 0.02-2.1, 0.02-2.0, 0.02-1.9, 0.02-1.8, 0.02-1.7, 0.02-1.6, 0.02-1.5, 0.02-1.4, 0.02-1.3, 0.02-1.2, 0.02-1.1, 0.02-1.0, 0.02-0.9, 0.02-0.8, 0.02-0.7, 0.02-0.6, 0.02-0.5, 0.02-0.4, 0.02-0.3, 0.02-0.2, 0.02-0.1, 0.02-0.09, 0.02-0.08, 0.02-0.07, 0.02-0.06, 0.02-0.05, 0.02-0.04, or 0.02-0.03 g/cm³. In some embodiments, membrane has a density between about 0.01-1.0, 0.02-1.0, 0.03-1.0, 0.04-1.0, 0.05-1.0, 0.06-1.0, 0.07-1.0, 0.08-1.0, 0.09-1.0, 0.1-1.0, 0.2-1.0, 0.3-1.0, 0.4-1.0, 0.5-1.0, 0.6-1.0, 0.7-1.0, 0.8-1.0, or 0.9-1.0 g/cm³.

In some embodiments, the final amount of pulp in the membrane is about 0.01 wt % to about 10 wt % (e.g., 0.01 to 0.1 wt %, 0.1 to 1.5 wt %, 0.1 to 2 wt %, 0.1 to 5 wt %, 1 to 10 wt %) of pulp (e.g., soft and/or hard wood pulp), wherein the wt % is calculated based on the dry mass, wherein the wt % is calculated based on the total weight of all solid components present in the slurry (and excludes the weight of the liquid components).

In some embodiments, a cellulosic membrane comprises 0.01-95 wt % of additive(s), wherein the wt % is calculated based on the dry mass, wherein the wt % is calculated based on the total weight of all solid components present in the slurry (and excludes the weight of the liquid components). For example, in some embodiments, a cellulosic membrane may comprise between 0.01% and 95% (e.g., between 0.01 and 90%, 0.01 and 80%, 0.01 and 70%, 0.01 and 60%, 0.01 and 50%, 0.01 and 40%, 0.01 and 30%, 0.01 and 20%, 0.01 and 10%, or 0.01 and 5%) wt % additive(s). In some embodiments, a cellulosic membrane comprises at least 0.01 wt % additive(s) (e.g., at least 0.01 wt %, 0.1 wt %, 0.5 wt %, 1 wt %, 5 wt %, 10 wt %, 15 wt %, 20 wt %).

In some embodiments, a cellulosic membrane comprises CNF and a wetting agent (e.g., a wetting mineral). In some embodiments, the ratio of CNF:wetting mineral present in a cellulosic membrane is about within a range of about 1:0.0001 to about 1:1000. In some embodiments, the ratio of CNF:wetting mineral present in a cellulosic membrane is within a range of about 1:0.0001-0.001, 1:0.001-0.1, 1:0.1-1, 1:1-5, 1:5-10, 1:10-20, 1:20-50, 1:50-100, or about 1:100-1000. In some embodiments, the ratio of CNF:wetting mineral present in a cellulosic membrane is about 1:0.0001, 1:001, 1:0.01, 1:0.2, 1:0.3, 1:0.4, 1:0.5, 1:0.6, 1:0.7, 1:0.8, 1:0.9, 1:0.1, 1:0.5, 1:1, 1:2, 1:3, 1:4, 1:5, 1:6, 1:10, 1:12, 1:14, 1:15, 1:20, 1:50, 1:100 or about 1:1000.

In some embodiments, the final ratio of pulp:CNF present in a cellulosic membrane is within a range of about 1:0.0001 to about 1:1000. In some embodiments, the ratio of pulp:CNF present in a cellulosic membrane is within a range of about 1:0.0001-0.001, 1:0.001-0.1, 1:0.1-1, 1:1-5, 1:5-10, 1:10-20, 1:20-50, 1:50-100, or about 1:100-1000. In some embodiments, the ratio of pulp:CNF present in a cellulosic membrane is about 1:0.0001, 1:001, 1:0.01, 1:0.2, 1:0.3, 1:0.4, 1:0.5, 1:0.6, 1:0.7, 1:0.8, 1:0.9, 1:0.1, 1:0.5, 1:1, 1:2, 1:3, 1:4, 1:5, 1:6, 1:10, 1:12, 1:14, 1:15, 1:20, 1:50, 1:100 or about 1:1000

In some embodiments, a membrane has a nanocellulose fiber solids content of about 0.01, 0.1, 0.5, 1, 5, 10, 15, 20, 25, 30, 35, 40, 45, 50, 55, 60, 65, 70, 75, 80, 85, 90, or 95 wt %. In some embodiments, a membrane has a nanocellulose fiber solids content of about 0.01 wt % to about 10 wt % (e.g., 0.01 to 0.1 wt %, 0.1 to 1.5 wt %, 0.1 to 2 wt %, 0.1 to 5 wt %, 1 to 10 wt %). In some embodiments, a membrane has a nanocellulose fiber solids content of about 1-90 wt %, 1-85 wt %, 1-80 wt %, 1-75 wt %, 1-70 wt %, 1-65 wt %, 1-60 wt %, 1-55 wt %, 1-50 wt %, 1-45 wt %, 1-40 wt %, 1-35 wt %, 1-30 wt % 1-25 wt %, 1-20 wt %, 1-15 wt %, 1-10 wt %, 1-9 wt %, 1-8 wt %, 1-7 wt %, 1-6 wt %, 1-5 wt %, 1-4 wt %, 1-3 wt %, or 1-2 wt %. In some embodiments, a membrane has a nanocellulose fiber solids content of about 0.01-95 wt %, 0.1-95 wt %, 5-95 wt %, 10-95 wt %, 15-95 wt %, 20-95 wt %, 25-95 wt %, 30-95 wt %, 35-95 wt %, 40-95 wt %, 45-95 wt %, 50-95 wt %, 55-95 wt %, 60-95 wt %, 65-95 wt %, 70-95 wt %, 75-95 wt %, 80-95 wt %, 85-95 wt %, 90-95 wt %, 91-95 wt %, 92-95 wt %, 93-95 wt %, or 94-95 wt %.

In some embodiments, a membrane of the present disclosure further comprises one or more additives. In some embodiments, one or more additives modify physical, mechanical or chemical properties of a membrane relative to an identical membrane lacking the one or more additives. In some embodiments, an additive comprises one or more foaming agents, blowing agents, and/or templating agents.

### Porosity

The membranes described herein, are characterized in their enhanced wicking ability. Porosity of membranes contributes to the wicking ability, and can be controlled and tuned by various methods, including the length drying time and method used to dry the cellulosic slurries. Amounts of the cellulosic materials within the membrane can also affect porosity (and also pore morphology). Pores that form lamellar-like channels have been shown to increase wicking ability.

In some embodiments, a membrane contains substantially homogenous porosity and/or pore size. In some embodiments, a membrane contains gradient of porosity and/or pore size.

In some embodiments, porosity of a membrane is within a range of at least 60-90%. In some embodiments, porosity of a membrane is within a range of at least 10-90%, 20-90%, 30-90%, 40-90%, 50-90%, 60-90%, 70-90%, or 80-90%. In some embodiments, porosity of a membrane is at least 10, 20, 30, 40, 50, 60, 70, 80, or at least 90%. In some embodiments, porosity of a membrane is determined by the bulk vs. absolute density of the membrane. In some embodiments, porosity and pore size distribution is tested using mercury porosimetry, or BET and/or BJH analysis.

In some embodiments, pores within a membrane have an average diameter between 1 nm-1000 microns (e.g., 10 nm-1000 microns, 100 nm-100 microns, 1-10 microns,). In some embodiments, pores within a membrane have an average diameter of between 1 nm-1000 nm (e.g., 1-10 nm, 10-20 nm, 20-100 nm, 100-200 nm, 200-300 nm, 300-400 nm, 400-500 nm, 500-600 nm, 600-700 nm 700-800 nm, 800-900 nm, 900-1000 nm). In some embodiments, pores within a membrane have an average diameter of between 1-1000 μm (e.g., 1-10 μm, 10-20 μm, 20-100 μm. 100-200 μm, 200-300 μm, 300-400 μm, 400-500 μm, 500-600 μm, 600-700 μm 700-800 μm, 800-900 μm, 900-1000 μm). In some embodiments, pore size/morphology can be determined investigated using scanning electron microscopy (SEM).

### Wicking

The present disclosure provides membranes comprising cellulosic materials that exhibit various improved characteristics, including improved wicking ability. Wicking generally can be understood as the ability of a liquid (e.g., a liquid sample containing an analyte) to be drawn through a material by capillary action.

Wicking can be measured, e.g., in wicking distance vs. time. In some embodiments, tests for wicking ability include vertical wicking tests, lateral wicking tests, or bidirectional wicking tests (where progression of a solvent, e.g., water, in the membrane by capillary action is measured). In some embodiments wicking is aided using a vacuum. In some embodiments, a bidirectional wicking tests include aid of a vacuum for testing wicking ability in one or more directions.

In some embodiments, speed is measured visually by viewing the progression of a solvent front of a liquid traveling through a membrane. In some embodiments, when a liquid reaches a certain point on the membrane, a visual indicator may appear.

In some embodiments, a visual indicator may be, for example, colorimetric labels (such as, for example, dyes, colloidal gold, and the like), fluorescent agents, chemiluminescent agents (such as, for example, acridinum esters, stabilized dioxetanes, and the like), and bioluminescent agents.

In some embodiments, a membrane is characterized by having the ability to wick a liquid through the membrane at a rate of at least 0.1 mm/s. In some embodiments, a membrane is able to wick a liquid through the membrane at a rate of at least 0.1, 0.2, 0.3, 0.4, 0.5, 1, 2, 3, 4, 5, 10, or 20 mm/s, or greater.

In some embodiments, improved wicking ability is an increase in wicking speed (e.g., in a vertical wicking test). In some embodiments, an increase in wicking speed is an increase of at least 0.1 mm/s compared to a membrane that does not include CNF. In some embodiments, an increase in wicking speed is an increase of at least 0.1, 0.2, 0.3, 0.4, 0.5, 1, 2, 3, 4, 5, 10, or 20 mm/s, or greater.

In some embodiments, wicking ability of a membrane is improved with respect to membranes without one or more of CNF, pulp, and wetting mineral. In some embodiments, wicking ability of a membrane with an additive is improved with respect to membranes without an additive.

### Analyte Immobilization

In some embodiments, one or more analytes in a liquid sample can be immobilized (i.e., detected) on a membrane.

In some embodiments, immobilization of an analyte is based on an interaction of an analyte in a fluid with a sensing agent. In some embodiments, a sensing agent contains a detectable entity. In some embodiments, a sensing agent is a chemically reactive species, such an enzyme, an antigen, or antibody.

Example enzymes include, for example, horseradish peroxidase (HRP), alkaline phosphatase, catalase, urease, and glucose oxidase.

Example detectible entities include, for example, various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes, chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available. Methods of measuring a detectable entity include, but are not limited to, visible detection, fluorescence, chemiluminescence, radioactivity, colorimetry, gravimetry, X-ray diffraction, X-ray absorption, magnetism, and enzymatic activity.

Types of analytes include e.g., pathogens, enzymes, immunologic mediators, nucleic acids, proteins, glycoproteins, lipopolysaccharides, protein adducts, tumor and cardiac markers, and/or low-molecular weight compounds, including, but not limited to, haptens, viruses or microorganisms, such as bacteria, fungi (e.g. yeast or molds) or parasites (e.g. amoebae or nematodes), immune mediators such as antibodies, growth factors, complement, cytokines, lymphokines, chemokines, interferons and interferon derivatives, C-reactive protein, calcitonin, amyloid, adhesion molecules, antibodies, and chemo-attractant components, drug molecules such as heroin or methamphetamine, and allergens.

In some embodiments, a liquid containing an analyte is a biological sample. In some embodiments, the biological sample is whole blood, serum, plasma, a mucous membrane fluid (of the oral, nasal, vaginal, anal, inner ear, and ocular cavities), cerebrospinal fluid (CSF), tear fluid, penile fluid, a secretion or exudate from a gland, or a secretion or exudate from a lesion or blister, e.g. lesions or blisters on the skin.

In some embodiments, an analyte is immobilized on the membrane in the form of aqueous droplets. In some embodiments, aqueous droplets are dried on the membrane at ambient and elevated temperature (30-70° C.) for applications such as ELISA tests based on enzyme-antigen-antibody interaction.

In some embodiments, an analyte is glucose, and a membrane is used for detecting glucose presence/levels in a biological sample.

In some embodiments, a membrane is used as a substrate in a lateral flow device/assay. In some embodiments, a membrane is used as a substrate in a diagnostic device.

In some embodiments, a membrane is used in a universal horizontal wicking substrate (e.g., a substrate compatible with multiple types of tests). In some embodiments, a membrane is used in a universal vertical wicking substrate.

In some embodiments, a membrane is used in an auto-sampling device (e.g., environmental testing strips). An auto-sampling device may be any type of a sampling device that does not require an external power source, such as a vacuum, electricity, or heat. In some embodiments, a membrane is used in a device for concentrating a biological or environment sample.

In some embodiments, provided membranes may be useful in filtering/separating one or more contaminants out of a solution (e.g., an aqueous solution). In some embodiments, a membrane has a removal capacity for a contaminant that is measured in milligram of contaminant per gram of membrane. In some embodiments, a contaminant is or comprises a physical, chemical, biological or radiological contaminant.

Examples of physical contaminants include e.g., sediment or organic material as a result of soil erosion. Examples of chemical contaminants include, e.g., nitrogen, bleach, salts, pesticides, metals, toxins produced by bacteria, and human or animal drugs. Examples of biological contaminants include, e.g., bacteria, viruses, protozoan, and parasites. Examples of radiological contaminants include, e.g., cesium, plutonium and uranium.

In some embodiments, a membrane is used in a water quality tests, e.g., testing one or more properties of a water sample, such as pH, alkalinity, chlorine content, and heavy metal ion content.

The present disclosure provides membranes comprising cellulosic materials that exhibit various improved characteristics, including one or more mechanical properties. In some embodiments, a mechanical property comprises flexure strength. In some embodiments, a mechanical property comprises compressive modulus. In some embodiments, a mechanical property comprises tensile strength.

### EXAMPLES

The following examples disclose exemplary cellulose membranes made from wood pulp, cellulose micro- and/or nano-fibers, and various additives, and methods of making and testing said cellulose membranes. The following examples are provided so as to describe to the skilled artisan how to make and use the membranes described herein and are not intended to limit the scope of the present disclosure.

### Example 1

In this Example, a membrane comprising one or more cellulosic materials (wood pulp and CNF) and a wetting agent was made and various physical and mechanical properties were tested. Membranes described in this Example were formed by (i) creating a cellulosic slurry by combining the cellulosic components and wetting agent with a liquid component; (ii) mixing the components of the cellulosic slurry and (iii) exposing the cellulosic slurry to a drying condition. Among the properties tested, a vertical and horizontal wicking test was performed on the resulting membranes. Membranes of the present Example were shown to have superior wicking and mechanical properties.

### Materials

The cellulosic materials used in this example include nanofibrillated cellulose (CNF) (soft and hardwood) and chemically bleached wood pulp (soft and hardwood).

CaCO₃ (ground CaCO₃, (G.C.C.) and precipitated CaCO₃ (P.C.C.) was used as the wetting agent.

### Preparation of the Cellulosic Slurry

A suspension of 0.1-5 wt % of the chemically bleached wood pulp (soft and hardwood) was prepared by soaking dry pulp in DI water for 24 hours followed by mechanical mixing for 30 minutes.

In a separate system, a suspension of 0.01-3 wt % aqueous CNF was mixed using mechanical mixing with the wetting mineral such that the ratio of the mass of CNF:wetting minerals was 1:1. Pulp and CNF/wetting mineral suspensions were mixed using the mixing techniques mentioned above, in such a way that the ratio of the pulp:CNF was 1:0.05, the ratio of CNF:Mineral was 1:1, and the ratio of pulp:wetting minerals was 1:0.01 to 0.3, respectively. The total solid content of the suspension was in the range of 0.5-1 wt % before dewatering step.

### Dewatering and Drying

The aqueous suspension of pulp, CNF and wetting mineral was then dewatered using gravity and vacuum filtration to generate a hydrogel of about 5-40% solid content. These hydrogels were then transferred to a drying unit for complete dehydration using an electrical oven at 105° C.

Membranes were tested for various properties, including porosity, wicking ability/analyte immobilization, and will be tested for other physical properties such including wetting mineral retention and tensile strength. Pulp membranes, CNF membranes, pulp+CNF membranes, CNF+CaCO₃ membranes were similarly prepared as controls.

To prepare CNF only membranes, freeze-drying method was used to dehydrate CNF suspensions since ambient or oven drying led to the formation of dense CNF films which were unusable for filtering purposes.

### Porosity and Pore Morphology

Porosity of the membranes was calculated from the bulk and absolute density of the membranes. The pore morphology was investigated using scanning electron microscopy (SEM). Porosity and pore size distribution will be tested via mercury porosimetry, BET and BJH analysis.

The dewatered and fully dried membranes showed a porosity in the range of 60-95%. The porous structure of the membranes is largely dependent on the CNF content. Membranes with low CNF content (˜0.1-2%) exhibit a lamellar type porous structure (see FIG. 1a), whereas the membranes with relatively higher CNF content (˜10-20%) exhibit a web like pore morphology (see FIG. 1b). For comparison, freeze-dried CNF only membranes were prepared. These membranes exhibited a web like interconnected porous morphology (FIG. 1c).

### Wicking Test

The wicking ability of the membranes were compared with other materials, including the CNF only membranes, pulp-only membranes, pulp+CNF membranes, and CaCO₃ membranes.

A vertical and lateral wicking test was performed on the membranes using an aqueous solution with a pH ranging from 4-10. A vertical wicking test was performed by submerging one end of the membrane, cut so that it is 50 mm in length, into a DI water bath (25° C.) with the other end of the sample fixed in a sample holder. The membrane was graduated every 5 mm to enable visualization of the solvent front and quantify the rate of solvent progression.

To test the unidirectional horizontal wicking properties of the membranes, water was introduced on top of the membrane, and the progression of the solvent front, over time, was recorded and used to calculate the wicking rate.

For bidirectional wicking determination, water was introduced on top of the membrane, and the progression of the solvent front was recorded. As the solvent front travelled to a specific distance in one direction, additional solvent (water) was introduced from the other end of the membrane. A vacuum/absorbent pad was also applied to the initial starting point of the membrane to facilitate backwards flow of water. The progression of the solvent front was recorded to calculate the backward wicking rate. A water-soluble dye was used to track the progression of the backwards flow.

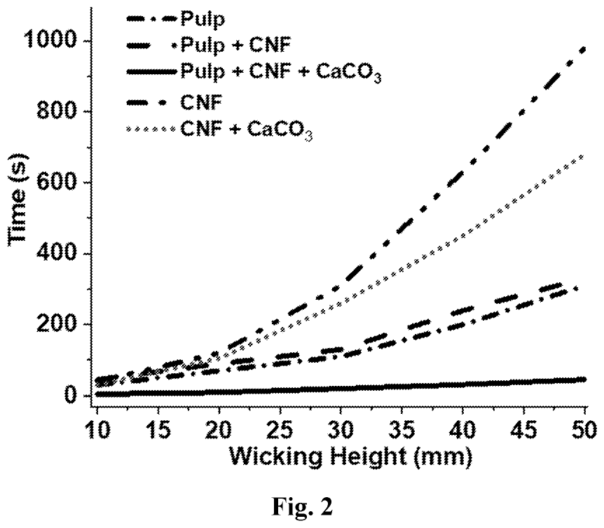

As shown in FIG. 2, the vertical wicking rate of the freeze-dried CNF membrane was very low (˜0.05 mm/s). Addition of 50% (w/w) wetting minerals (CNF+CaCO₃ membranes), slightly improved the wicking performance (0.08 mm/s).

In contrast, when wood pulp was used as the primary matrix component, a highly porous structure is achieved regardless of the drying methods. These materials exhibited ˜3 times higher wicking rate compared to the CNF membranes. When CNF is incorporated to these pulp membranes, the wicking rate started to exhibit a decreasing trend since CNF induced a web like interconnected porous network, rather than a lamellar pore morphology. Without wishing to be bound by theory, a decrease in wicking rate with the addition of CNF could be due to a decreased total internal pore volume in the membrane.

Surprisingly, the addition of wetting minerals to the pulp membranes did not improve the wicking performance of the membranes. The possible reason for this phenomenon could be the lack of retention of wetting minerals in the membrane during the dewatering step in the membrane fabrication process. In contrast, CNF exhibited high retention of CaCO₃ during the dewatering process, possibly due to high surface area and wettability of the CNF materials. The high retention was observed during the wicking test. Further analysis using an Ash Test will determine the amount of wetting mineral retained in the membrane.

When CNF and CaCO₃ was incorporated into a pulp suspension (to yield the above described “Pulp+CNF+CaCO₃ as denoted in FIG. 2). High retention of the wetting minerals was observed, which will be confirmed by an Ash Test. The wicking rate of the membrane (Pulp+CNF+CaCO₃) increased surprisingly (˜20 times greater as compared to the CNF-only membrane, and ˜7 times greater than the pulp-only membranes). This phenomenon suggests that CNF acts as a binding and immobilizing/dispersing/stabilizing agent for the wetting minerals in the pulp suspensions.

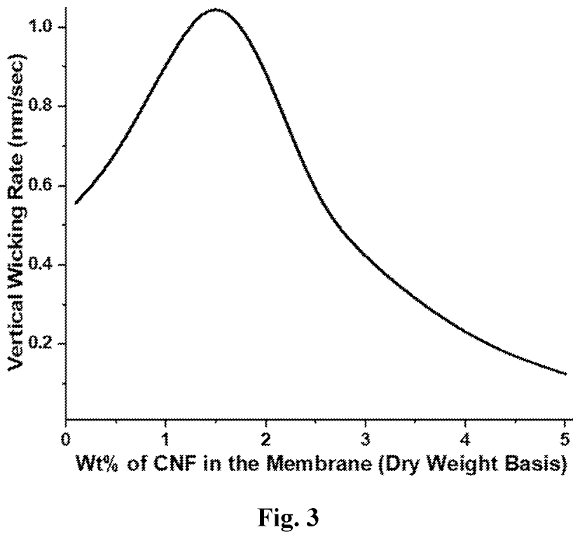

The addition of a significant amount of wetting minerals would require a large amount of CNF in the membrane matrix. However, the largest value of wicking rate was exhibited when CNF was present in the membrane in the range of 1-2 wt % (by dry mass basis). Below this range, there is no significant effect on the wicking rate of the pulp+CNF+CaCO₃ membranes due to low retention of wetting minerals. Above this range of CNF fractions, due to the enhancement of web like pore network, and reduction of total porosity, the wicking rate decreases dramatically. Therefore, 1-2 wt % (dry mass basis) is the optimum range of CNF fraction in these pulp membrane matrices for superior wicking rate. When 1-2 wt % CNF is used, the maximum fraction of CaCO₃ retained in the matrices was about 10-20 wt % (see FIG. 3).

### Analyte Immobilization

Enzymes (sensing agents) were immobilized on the CNF+pulp+CaCO₃ membrane in the form of aqueous droplets followed by drying at ambient and elevated temperature (30-70° C.).

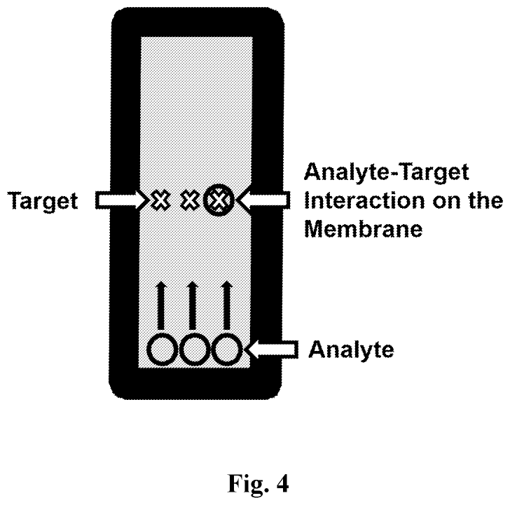

A solution containing an antibody (analyte) and an indicator (3,3′,5,5′-Tetramethylbenzidine (TMB)) was tested by unidirectional flow on the membrane containing a sensing agent (HRP enzyme) and an antigen recognized by the antibody (see setup in FIG. 4). The sensing procedure using CNF+pulp+CaCO₃ membrane for detecting a specific analyte (antibody) was completed in ˜3 minutes (i.e., from the time the analyte-containing solution was applied to the membrane to the time the indicator was observed). In the bidirectional procedure, the sensing process took about 7 minutes for the completion.

### Tensile Strength Test

In addition to the enhanced wicking properties, addition of the CNF to the wood pulp membrane enhanced the tensile strength of the membrane. This was demonstrated when the wet membranes were transferred from the dewatering unit (capillary dewatering) to the drying unit without any visible damage (break or fracture). In membranes without CNF, cracking and tearing was observed when transferring the membranes from the dewatering unit to the drying unit.

Dynamic mechanical analysis (DMA) will be conducted on the dry membranes for the tensile strength test.

### Wetting Minerals Retention Ash Test

An ash test and atomic absorption spectroscopy will be conducted to calculate the residual minerals in the membranes.

A leaching test will also be performed to test loss of minerals in wet conditions.

### EQUIVALENTS

It is to be appreciated by those skilled in the art that various alterations, modifications, and improvements to the present disclosure will readily occur to those skilled in the art. Such alterations, modifications, and improvements are intended to be part of the present disclosure, and are intended to be within the spirit and scope of the invention. Accordingly, the foregoing description and drawing are by way of example only and any invention described in the present disclosure if further described in detail by the claims that follow.

Those skilled in the art will appreciate typical standards of deviation or error attributable to values obtained in assays or other processes as described herein. The publications, websites and other reference materials referenced herein to describe the background of the invention and to provide additional detail regarding its practice are hereby incorporated by reference in their entireties.

1. A membrane comprising a porous matrix material, wherein the porous matrix material comprises:
(i) wood pulp;
(ii) cellulose nanofilbrils (CNF); and
(iii) one or more wetting minerals.

2. The membrane of claim 1, wherein the one or more wetting minerals comprises calcium carbonate (CaCO₃), TiO2, Alumina, Fiberglass, or a combination thereof.

3. The membrane of claim 1 or 2, wherein the CNF is present in a concentration within the range of 0.1 to 1.5 wt % based on dry mass basis.

4. The membrane of any one of claims 1-3, wherein the one or more wetting minerals are present in a concentration within the range of 0.1 to 20 wt % of the porous matrix material.

5. The membrane of any one of claims 1-4, wherein the CNF comprises CNF obtained by TEMPO (2,2,6,6-tetramethylpiperidine-1-oxyl radical)-mediated oxidation.

6. The membrane of any one of claims 1-5, wherein when contacted with a fluid comprising an analyte, the analyte solution travels across the membrane through capillary action.

7. The method of claim 6, wherein the analyte is immobilized on a specific site of the membrane.

8. The membrane of claim 6, wherein the analyte travels across the membrane at a rate of greater than about 0.5 mm per second.

9. The membrane of any one of claims 6-8, wherein the analyte is or comprises a biological material.

10. The membrane of any one of claims 1-9, wherein the porous matrix material is substantially homogeneous.

11. The membrane of any one of claims 1-10, wherein the porous matrix material comprises a porosity of at least 60-90%.

12. The membrane of any one of claims 1-11, wherein the porous matrix material comprises one or more additives.

13. The membrane of claim 11, wherein the one or more additives comprises a foaming agent, a blowing agent, a templating agent, a plasticizer, or a combination thereof.

14. The membrane of claim 12 or 13, wherein the one or more additives are present in a concentration within the range of 0.1 to 10 wt % based on dry mass basis.

15. The membrane of claim 13, wherein the foaming agent comprises a surfactant.

16. The membrane of claim 15, where the surfactant comprises glucosides and/or myristic acid.

17. The membrane of claim 15, wherein the surfactant comprises a biosurfactant such as fungi, bacteria, yeast, glycolipids, phospholipids, glycopeptides, saponins, fatty acids, proteins, polysaccharides or a combination thereof.

18. The membrane of claim 13, wherein the blowing agent comprises sodium bicarbonate.

19. The membrane of claim 13, wherein the templating agent comprises salt, ice, dry ice or a combination thereof.

20. The membrane of claim 13, wherein the plasticizer comprises acetylated monoglycerides, alkyl citrates, epoxidized soybean oil, proteins, polyethylene glycol, fatty acids, or combinations thereof.

21. A method comprising:
(i) providing slurry comprising wood pulp and water;
(ii) mixing cellulose nanofilbrils (CNF) and one or more wetting minerals into the slurry; and
(iii) drying the slurry to form a porous matrix material.

22. The method of claim 21, wherein the one or more wetting minerals comprises calcium carbonate (CaCO₃), TiO2, Alumina, Fiberglass, or a combination thereof.

23. The method of claim 21 or 22, wherein drying the slurry comprises capillary dewatering, infrared drying, lyophilization, and/or microwave irradiation.

24. The method of any one of claims 21-23, wherein the concentration of CNF is 0.1 to 1.5 wt % of the porous matrix material.

25. The method of any one of claims 21-24, wherein the one or more wetting minerals are present in a concentration within the range of 0.1 to 20 wt % of the porous matrix material.

26. A method of separating an analyte from a fluid comprising:
(i) providing a membrane comprising a porous matrix material; and
(ii) contacting the membrane with a fluid comprising an analyte so that the fluid enters the membrane through capillary action, thereby separating the analyte;
wherein the porous matrix material is a composite material that comprises wood pulp, CNF, and one or more wetting minerals.

27. The method of claim 26, wherein the one or more wetting minerals comprises calcium carbonate (CaCO₃), TiO2, Alumina, Fiberglass, or a combination thereof.

28. The method of claim 26 or 27, wherein the contacting step is or comprises contacting the membrane with a fluid contained in an adjacent space or adjacent material.

29. The method of any one of claims 26-28, wherein the fluid travels across the membrane.

30. The method of claim 29, wherein the fluid passively travels across the membrane.

31. The method of claim 29, wherein the fluid travels across the membrane with the aid of a vacuum or positive pressure on the fluid.

32. The method of any one of claims 26-31, wherein the analyte is immobilized on the membrane.

33. The method of claim 32, wherein the immobilized analyte is or comprises a biological material.
